(19) United States
(12) Patent Application Publication
(10) Pub. No.: US 2017/0319818 A1
(43) Pub. Date: Nov. 9, 2017

(54) DRUG DELIVERY DEVICE

(71) Applicant: Cook Medical Technologies LLC, Bloomington, IN (US)

(72) Inventors: Angela R. Dowell, Lafayette, IN (US); Keith Milner, West Lafayette, IN (US)

(21) Appl. No.: 15/586,470

(22) Filed: May 4, 2017

Related U.S. Application Data
(60) Provisional application No. 62/332,719, filed on May 6, 2016.

(51) Int. Cl. A61M 25/00; A61B 17/22; A61M 25/09

(52) U.S. Cl. CPC A61M 25/007; A61M 25/09; A61M 25/0023; A61M 25/0045; A61M 25/008; A61B 17/22; A61M 2025/0046; A61M 2025/09141; A61M 2202/0413; A61M 2205/0266

(57) ABSTRACT

The present disclosure provides for a fluid delivery device for a body vessel and a method of use. The fluid delivery device may have an elongate member having a proximal end extending along the longitudinal axis to a closed distal end. The elongate member may also have an inner wall defining a fluid delivery lumen and a wire guide lumen. The elongate member may have a tapered region and a distal portion between the proximal and distal ends. The device may have a first state for device delivery to the treatment site.

### BACKGROUND

### 1. Field of the Invention

The present disclosure relates to medical devices. More particularly, the disclosure relates to a medical device for locally delivering a fluid or drug to a treatment site.

### 2. Background

Various intraluminal procedures require the infusion of drugs to treat physiological conditions, such as those in the vasculature at a vessel wall (e.g. restenosis). This may be done, for example, with a drug-coated inflation balloon. During the procedure, the balloon may be inflated at the treatment site so that the outer surface of the balloon is pressed against the vessel wall. In this manner, the drug may be pressed against the treatment site and absorbed by the wall tissue.

While this approach has the advantage of applying the drug directly to the tissues where treatment is desired, it can be difficult to achieve the desired level of treatment since the balloon may block blood flow in the vessel. Conventional balloons fill the vessel lumen when inflated and block blood flow through the vessel. In such a case, the balloon can only remain inflated for a short period of time. Blood flow can typically be blocked for only a short period of time (e.g., usually less than 3 minutes) in order to prevent starving downstream tissues of blood and oxygen.

Drug delivery time can be increased by delivery near the desired treatment site, flowing through the patient's vascular system (without an inflation balloon). However, a significant portion of the drug may not interact with the desired treatment site and may flow downstream away from the treatment site, causing disadvantages. Accordingly, the inventors believe that there is a need for further drug delivery devices.

### BRIEF SUMMARY OF THE INVENTION

The invention may include any of the following embodiments in various combinations and may also include any other aspect described below in the written description or in the attached drawings. The medical device provided for in this disclosure may deliver a fluid to a body vessel, having a vessel wall. In one embodiment, the device may have an elongate member having a proximal end extending along a longitudinal axis to a distal end.

The distal end may form an atraumatic tip. The elongate member may have an elongate length from the proximal end to the distal end, the elongate length being about 100 centimeters.

The elongate member may also have an inner wall disposed at the proximal end and distally extending to the distal end. The inner wall may define a fluid delivery lumen and a wire guide lumen. More specifically, the wire guide lumen may be within the fluid delivery lumen. Additionally, the elongate member may have an outer wall formed to close the fluid delivery lumen at the distal end such that no fluid may exit the distal end.

The elongate member may also have a tapered region between the proximal and distal ends. The tapered region may have a tapered diameter distally decreasing along the longitudinal axis such that the fluid delivery lumen has a first cross-sectional area proximal the tapered region and a second cross-sectional area distal the tapered region. The first cross-sectional area may be greater than the second cross-sectional area. The first cross-sectional area may have a first circumference about three to about nine French.

The wire guide lumen may have a third cross-sectional area and an inner diameter being constant along the longitudinal axis. The third cross-sectional area may be smaller than the second cross-sectional area.

The elongate member may have a distal portion distally extending from the tapered region to the distal end and having a plurality of holes formed therethrough. The distal portion having a portion length being about 5 centimeters. The distal portion may be non-spiral in a first state for device delivery and may be spiral in a second state for fluid delivery. The spiral may have a pitch being about twenty-six (26) millimeters.

Each hole of the plurality of holes may be equidistant from all adjacent holes. In the second state, the plurality of holes may be formed in linear rows extending along the longitudinal axis arranged to contact the vessel wall. Each hole may have a hole diameter being 0.002 inches.

The taper region may have or be formed from a first material with a first durometer. The distal portion may have or be formed from a second material with a second durometer, the second durometer being less than the first durometer. The first cross-sectional area may have a first flow rate therethrough, and the second cross sectional area may have a second flow rate therethrough such that the first flow rate may be greater than the second flow rate.

The device may further include a wire guide disposed in the wire guide lumen. One of the wire guide and the distal portion may have a shape-memory material such that the distal portion moves between the first state and the second state. This arrangement may provide fluid delivery from the fluid delivery lumen and through the holes of the distal portion to the vessel wall. The shape-memory material may be Nitinol, which may be heat-set.

In another embodiment, the elongate member may not have a taper region. Instead, the elongate member may have the proximal end extending to a distal portion, which distally extends to the distal end. The elongate member may have the inner wall defining the wire guide lumen being within the fluid delivery lumen. The fluid delivery lumen may be closed at the distal end by the outer wall.

The present disclosure also provides for a method of use. The method may deliver a fluid to a body vessel by (1) disposing a medical device in the body vessel, the device having any or all of the features discussed herein; (2) moving the wire guide in one of a proximal direction and a distal direction to move the distal portion from the first state to the second state; and (3) delivering a fluid through the fluid delivery lumen and through the plurality of holes of the distal portion to the vessel wall.

The step of moving the wire guide may include moving the wire guide in the proximal direction to move the distal portion from the first state to the second state. The step of moving the wire guide may include moving the wire guide in the distal direction to move the distal portion from the first state to the second state. The step of delivering a fluid through the fluid delivery lumen comprises delivering tissue plasminogen activator (tPA). Other steps and order of steps are also possible.

As one possible advantage of the above described embodiments and arrangements, the device may allow localized delivery of a fluid or drug to a treatment site at a vessel wall, while not occluding the vessel. Therefore, the vessel remains open to blood flow, increasing possible treatment times.

The present disclosure may be better understood by referencing the accompanying figures.

### BRIEF DESCRIPTION OF THE DRAWINGS

FIGS. 1A-D depict side view of a medical device in accordance with embodiments of the present invention.

FIGS. 2A-B depict cross-sectional view of the device of FIG. 1A.

FIG. 3 is a partial, environmental view of the device of FIG. 1A.

FIGS. 4A-B are partial side views of the medical device of FIG. 1A.

FIG. 5 is a partial, cross-sectional view of the device of FIG. 1A.

FIG. 6 is a flow diagram of a method of use in accordance with one embodiment of the present invention.

### DETAILED DESCRIPTION

The present disclosure will now be described more fully with reference to the accompanying figures, which show preferred embodiments. The accompanying figures are provided for general understanding of the structure of various embodiments. However, this disclosure may be embodied in many different forms. These figures should not be construed as limiting and they are not necessarily to scale.

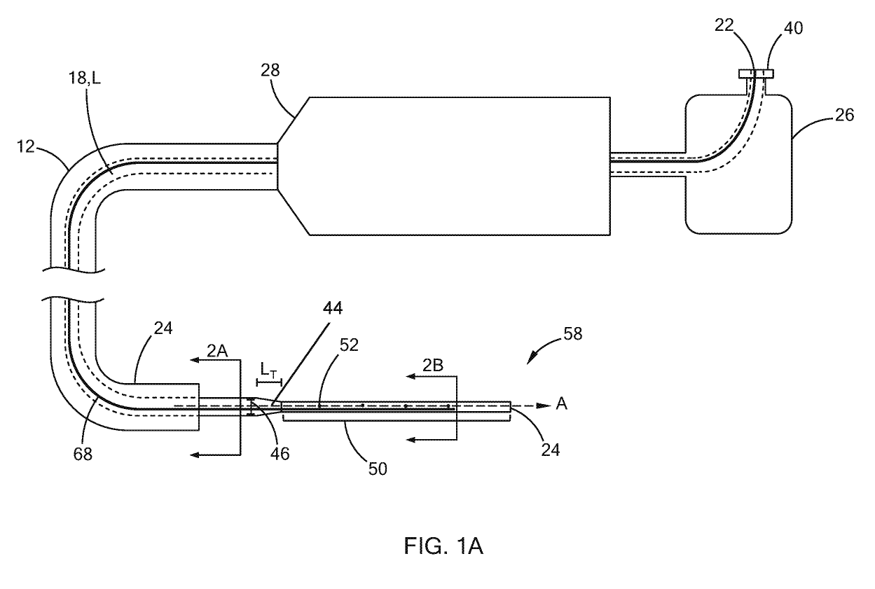

FIG. 1A depicts the device being inserted through an outer or introducer sheath 12 through port 40 in a first handle member 26. The introducer sheath 12 may also have a second handle member 28 to manipulate the sheath 12.

In FIG. 1A, the fluid delivery device may comprise an elongate member 18 having a proximal end 22 extending along a longitudinal axis A to a distal end 24.

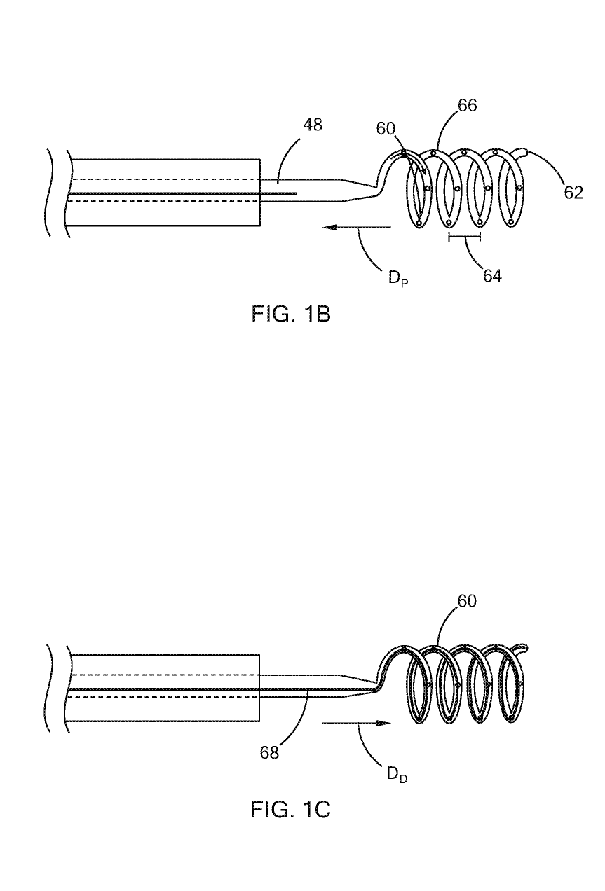

The elongate member 18 may have an elongate length L from the proximal end 22 to the distal end 24. In some embodiments, the elongate length L may be about 100 cm. “About” or “substantially” mean that a given quantity is within 10%, preferably within 5%, more preferably within 1%. The elongate member 18 may also have an inner wall (discussed further in FIGS. 2A-B) disposed at the proximal end 22 and distally extending to the distal end 24.

Although not visible in FIG. 1A, the inner wall may define a wire guide lumen and a fluid delivery lumen within the elongate member 18. The elongate member 18 may have an outer wall disposed at the distal end 24, which may be formed to close the fluid delivery lumen, blocking fluid from exiting the distal end 24. One skilled in the art will understand that a closed distal end 24 of the fluid delivery lumen may allow a higher fluid pressure inside the elongate member 18 than if the outer wall did not close the fluid delivery lumen. It is also possible to form the device without the inner wall, and having a single lumen for the wire guide and fluid delivery. Further details of the lumens will be discussed in FIGS. 2A-B.

The elongate member 18 may also have a tapered region 44. The tapered region 44 may have a tapered diameter 46 distally decreasing along the longitudinal axis A such that the fluid delivery lumen has a first cross-sectional area (2A) proximal the taper region 44 and a second cross-sectional area (2B) distal the taper region 44.

The elongate member 18 may have a distal portion 50 distally extending from the tapered region 44 to the distal end 24. The distal portion 50 may have a plurality of holes 52 formed therethrough. FIG. 1A depicts the distal portion in a first state 58. The distal portion 50 may be non-spiral, or straight, in the first state 58. FIGS. 1B-1C depict the device in a second state 60. The distal portion 50 may have a spiral 62 in the second state 60 for fluid delivery.

The device may further comprise a wire guide 68 disposed in the wire guide lumen. It will be understood that even though the wire guide 68 is visible in FIGS. 1A-C, it may be occluded by the outer sheath and elongate member 18 in practice. One of the wire guide 68 and the distal portion 50 may comprise a shape-memory material (e.g. Nitinol) such that the distal portion 50 moves between the first state 58 and the second state 60. In this way, the device provides fluid delivery from the fluid delivery lumen and through the holes 52 of the distal portion 50 to the vessel wall.

Nitinol is a metal alloy of nickel and titanium having unique shape memory setting properties and being biocompatible. At a transition temperature, Nitinol may undergo a phase change from Martensite to Austenite, changing its structure. In addition to this phase change ability, Nitinol is also quite flexible.

As shown in FIGS. 1A-C, the device may have two configurations. In a first configuration, the distal portion 50 may comprise the shape-memory material, possibly being Nitinol. In this configuration, the practitioner may insert the wire guide 68 into the wire guide lumen to the distal end 24. The wire guide 68 is of a sufficient stiffness to straighten the shape-memory material of the distal portion 50 into the first state 58, as shown in FIG. 1A.

In FIG. 1B, when the distal portion is that the treatment site, the practitioner may move the wire guide 68 in a proximal direction DP, as shown. Moving the wire guide 68 in the proximal direction DP allows the distal portion to form spiral 62, having a pitch 64. The helix or spiral 62 forms the second state 60.

In a second configuration, the distal tip 50 may not contain the shape-memory material to form a spiral. Instead, the wire guide 68 may contain the shape-memory material (e.g. Nitinol) to form a spiral. As shown in FIG. 1C, now the practitioner may move the wire guide and a distal direction Do to move the distal portion from the first state (FIG. 1A, 58) to the second state 60. This configuration may involve the use of a separate wire guide for tracking to the treatment site before using the wire guide 68 to form the second state 60.

In either configuration, the part of the device comprising the shape-memory material may change from martensite to austenite configuration by a change in temperature (i.e. body temperature) or by restricting the part comprising the shape-memory material, such as by constricting the shape-memory material with the stiffness of the other of the wire guide 68 or the distal portion 50 (i.e. constricting the shape-memory material with whichever part does not contain the shape-memory material).

In either configuration, the distal portion 50 may have a change in material from the rest of the elongate member such that it forms a floppy or flexible distal portion 50. This flexible portion may better accommodate the change from the first state to the second state. In some embodiments, the distal portion 50 may have a portion length being about 5 centimeters (cm) to about 10 centimeters. In one example, the length may be 8 centimeters.

For example in FIG. 1B, the taper region may comprise a first material 48 having a first durometer. The distal portion may comprise a second material 66 having a second durometer. The second durometer may be less, or more flexible, than the first durometer to form the floppy tip.

In FIG. 1D, the elongate member may not have a taper region. Instead, the elongate member may have a uniform diameter along its length. The diameter may change at the distal portion. With or without a taper region, the distal portion may move between its two configurations as described in FIGS. 1B-C.

The device may further comprise a lubricious coating disposed about the distal portion 50 and/or the distal end 24. In addition, the distal end 24 may comprise a rounded or atraumatic tip. A skilled artisan will understand that such a coating and/or an atraumatic tip may facilitate device delivery to the treatment site without damage to the vessel wall.

Lines 2A and 2B of FIG. 1A are shown in cross-sectional views of FIGS. 2A-B, respectively. These cross-sections show further details of the fluid delivery lumen 30 and the wire guide lumen 28. In FIG. 2A, the inner wall 26 defines the wire guide lumen 28 being within the fluid delivery lumen 30. The fluid delivery lumen 30 has a first cross-sectional area 32 distal the taper region. The fluid delivery lumen 30 also has a first circumference 34. The first circumference 34 may be about three to about nine French.

In FIG. 2B, the fluid delivery lumen has a second cross-sectional area 38 distal the taper region. The second cross-sectional area 38 is smaller than the first cross-sectional area 32. In addition the wire guide lumen 28 has a third cross-sectional area 70. The third cross-sectional area 70, and its corresponding inner diameter, may be constant along the longitudinal axis. The third cross-sectional area 70 may be smaller than the second cross-sectional area 38.

As discussed above, an outer wall may be formed to close the fluid delivery lumen 30 at the distal end. Contrastingly, the wire guide lumen 28 may be open at the distal end to allow the wire guide to pass through. Alternatively, the wire guide lumen 28 may be closed at the distal end by the outer wall such that the wire guide may only advance to the distal end.

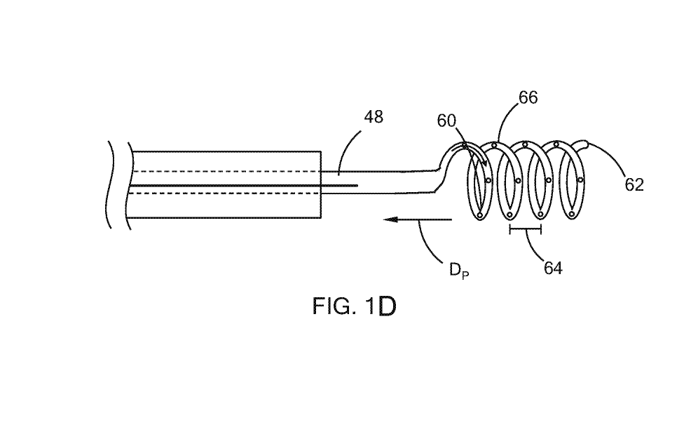

FIG. 3 is an environmental view of a portion of the device, depicting the second state with fluid delivery 16 of a fluid F. In this view, the first cross-sectional area proximal the taper region has a first flow rate 36 of the fluid F. A skilled artisan will understand that the fluid F may be any intended delivery fluid, including tissue plasminogen activator (tPA). tPA is an enzyme utilized to breakdown blood clots. tPA is often delivered directly to the site of an occlusion in a vessel. It may be utilized to treat various conditions, such as pulmonary embolism, myocardial infarction, and stroke.

Because of the taper region, the second cross-sectional area distal the taper region may have a second flow rate 42 therethrough. The first flow rate 36 may be greater than the second flow rate 42. Because of this change in flow rate, the taper region may allow the practitioner to deliver a fluid F with a lower fluid pressure along the elongate length to the taper region. When the fluid reaches the taper region, the fluid pressure may increase such that the fluid flow rate 42 slows down from the fluid flow rate 36, giving the practitioner greater control of the fluid flow to the vessel wall 14.

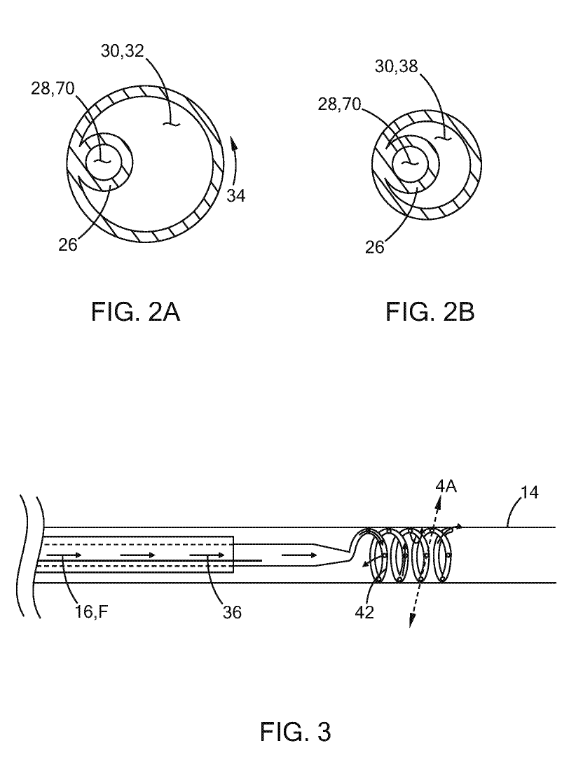

Line 4A is shown in FIG. 4A, depicting the plurality of holes 52. As shown in FIG. 4A, each hole of the plurality of holes 52 may be equidistant or equally spaced from all adjacent holes. “Adjacent” will be used herein to refer to an immediately neighboring hole. Each hole may have a hole diameter of about 0.002 inches.

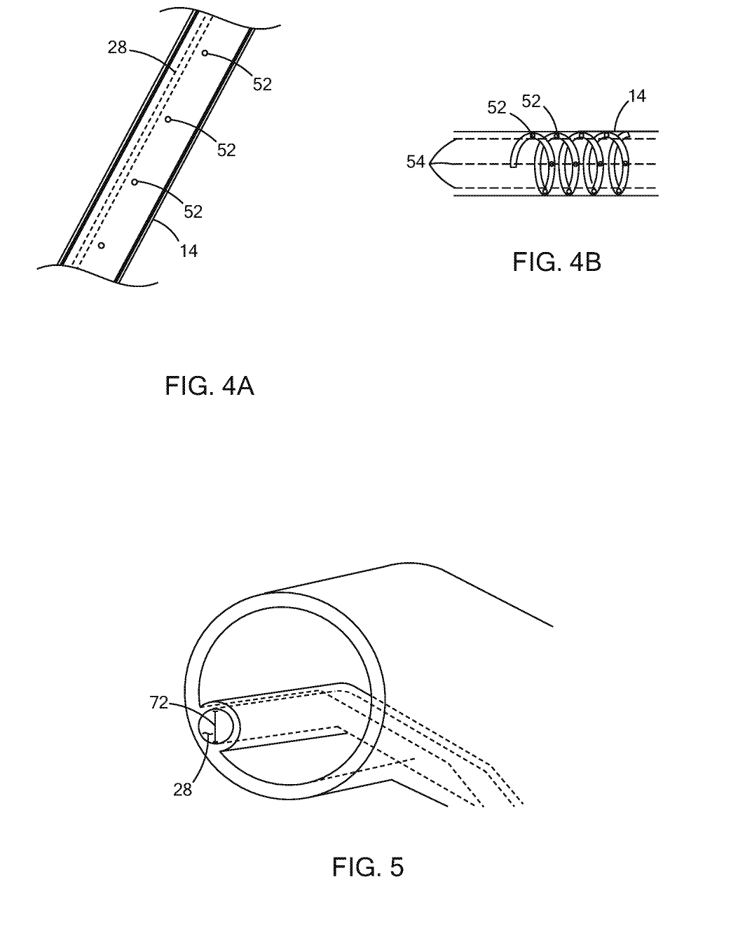

FIG. 4B additionally shows that the plurality of holes 52 may be formed in longitudinal rows 54 extending along the longitudinal axis arranged to contact the vessel wall 14 in the second state (i.e. helical state). In other words, the holes 52 may be disposed about the distal portion such that when the distal portion is in the second state, the holes are outwardly arranged to contact the vessel wall (and not inwardly disposed to face a vessel lumen). Such an arrangement may involve the holes 52 forming longitudinal rows 54. FIG. 5 depicts another partial, cross-sectional view of the elongate member, forming the wire guide lumen 28 having an inner diameter 72.

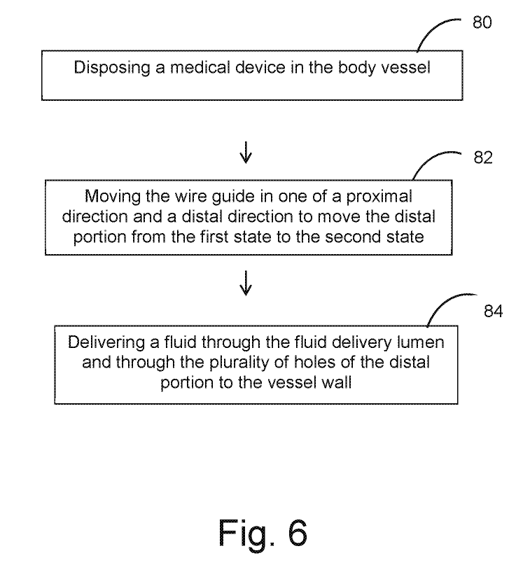

FIG. 6 depicts a flow diagram for steps of a method of fluid delivery in the body vessel. In step 80, the practitioner may dispose a medical device in the body vessel, the device having any or all of the features discussed herein. In step 82, the practitioner may move the wire guide in one of a proximal direction and a distal direction to move the distal portion from the first state to the second state. In step 84, the practitioner may deliver a fluid through the fluid delivery lumen and through the plurality of holes of the distal portion to the vessel wall.

In this manner that the practitioner may use the device to locally deliver a fluid or drug to an intended treatment site of the vessel wall. It will be understood that even though the fluid may be delivered to the vessel wall, some fluid may be allowed to flow into the vessel lumen with the blood flow. The practitioner may desire to deliver the fluid in a bolus of fluid or a steady stream of fluid delivery. Additionally, the step of delivering a fluid through the fluid delivery lumen may include delivering tPA.

It should be understood that the foregoing relates to exemplary embodiments of the disclosure and that modifications may be made without departing from the spirit and scope of the disclosure as set forth in the following claims. While the disclosure has been described with respect to certain embodiments it will be appreciated that modifications and changes may be made by those skilled in the art without departing from the spirit of the disclosure.

1. A device for fluid delivery in a body vessel having a vessel wall, the device comprising:
an elongate member having a proximal end extending along a longitudinal axis to a distal end, the elongate member having an inner wall disposed at the proximal end and distally extending to the distal end, the inner wall defining a fluid delivery lumen and a wire guide lumen, the elongate member having a tapered region between the proximal and distal ends, the tapered region having a tapered diameter distally decreasing along the longitudinal axis such that the fluid delivery lumen has a first cross-sectional area proximal the tapered region and a second cross-sectional area distal the tapered region, the first cross-sectional area being greater than the second cross-sectional area, the elongate member having a distal portion distally extending from the tapered region to the distal end and having a plurality of holes formed therethrough, the distal portion being non-spiral in a first state for device delivery and being spiral in a second state for fluid delivery; and
a wire guide disposed in the wire guide lumen, one of the wire guide and the distal portion comprising a shape-memory material such that the distal portion moves between the first state and the second state, providing fluid delivery from the fluid delivery lumen and through the holes of the distal portion to the vessel wall.

2. The device of claim 1 wherein the inner wall defines the wire guide lumen being within the fluid delivery lumen, the elongate member having an outer wall formed to close the fluid delivery lumen at the distal end.

3. The device of claim 1 wherein the wire guide lumen has a third cross-sectional area and an inner diameter being constant along the longitudinal axis.

4. The device of claim 3 wherein the third cross-sectional area is smaller than the second cross-sectional area.

5. The device of claim 1 wherein the wire guide comprises the shape-memory material being Nitinol.

6. The device of claim 1 wherein the distal portion comprises the shape-memory material being Nitinol.

7. The device of claim 1 wherein the taper region comprises a first material with a first durometer and the distal portion comprises a second material with a second durometer, the second durometer being less than the first durometer.

8. The device of claim 1 wherein the first cross-sectional area has a first flow rate therethrough and the second cross sectional area has a second flow rate therethrough, the first flow rate being greater than the second flow rate.

9. The device of claim 1 wherein each hole of the plurality of holes is equidistance from all adjacent holes.

10. The device of claim 1 wherein the plurality of holes being formed in longitudinal rows arranged to contact the vessel wall in the second state.

11. The device of claim 1 wherein the elongate member has an elongate length from the proximal end to the distal end, the elongate length being about 100 centimeters, the distal portion having a portion length being about 5 centimeters.

12. The device of claim 1 wherein each hole of the plurality of holes has a hole diameter being about 0.002 inches.

13. The device of claim 1 wherein the spiral of the distal portion has a pitch being about 26 millimeters.

14. The device of claim 1 wherein the first cross-sectional area has a first circumference about three to about nine French.

15. The device of claim 1 further comprising a lubricious coating disposed about the distal portion and the distal end.

16. The device of claim 1 wherein the distal end comprises an atraumatic tip.

17. A device for fluid delivery in a body vessel having a vessel wall, the device comprising:
an elongate member having a proximal end extending along a longitudinal axis to a distal end, the elongate member having an inner wall disposed at the proximal end and distally extending to the distal end, the inner wall defining a fluid delivery lumen and a wire guide lumen being within the fluid delivery lumen, the elongate member having an outer wall formed to close the fluid delivery lumen at the distal end and having a distal portion distal the proximal end, the distal portion extending to the distal end and having a plurality of holes formed therethrough, the distal portion being non-spiral in a first state for device delivery and being spiral in a second state for fluid delivery; and
a wire guide disposed in the wire guide lumen, one of the wire guide and the distal portion comprising a shape-memory material such that the distal portion moves between the first state and the second state, providing fluid delivery from the fluid delivery lumen and through the holes of the distal portion to the vessel wall.

18. A method of fluid delivery in a body vessel having a vessel wall, the method comprising:
disposing a medical device in the body vessel, the device comprising;
an elongate member having a proximal end extending along a longitudinal axis to a distal end, the elongate member having an inner wall disposed at the proximal end and distally extending to the distal end, the inner wall defining a fluid delivery lumen and a wire guide lumen, the elongate member having a tapered region between the proximal and distal ends, the tapered region having a tapered diameter distally decreasing along the longitudinal axis such that the fluid delivery lumen has a first cross-sectional area proximal the tapered region and a second cross-sectional area distal the tapered region, the first cross-sectional area being greater than the second cross-sectional area, the elongate member having a distal portion distally extending from the tapered region to the distal end and having a plurality of holes formed therethrough, the distal portion being non-spiral in a first state for device delivery and being spiral in a second state for fluid delivery; and
a wire guide disposed in the wire guide lumen, one of the wire guide and the distal portion comprising a shape-memory material such that the distal portion moves between the first state and the second state, providing fluid delivery from the fluid delivery lumen and through the holes of the distal portion to the vessel wall;
moving the wire guide in one of a proximal direction and a distal direction to move the distal portion from the first state to the second state; and
delivering a fluid through the fluid delivery lumen and through the plurality of holes of the distal portion to the vessel wall.

19. The method of claim 17 wherein the step of moving the wire guide comprising moving the wire guide in the proximal direction to move the distal portion from the first state to the second state.

20. The method of claim 17 wherein the step of moving the wire guide comprising moving the wire guide in the distal direction to move the distal portion from the first state to the second state.
